(19) United States
(12) Patent Application Publication
(10) Pub. No.: US 2025/0336285 A1
(43) Pub. Date: Oct. 30, 2025

(54) NOTIFICATION METHOD

(71) Applicant: NEC Corporation, Minato-ku, Tokyo (JP)

(72) Inventors: Yuan LUO, Tokyo (JP); Mitsuru NOMA, Tokyo (JP); Osamu HISAMATSU, Tokyo (JP); Akihiko SHIBANO, Tokyo (JP); Hiroaki KATAOKA, Tokyo (JP); Kosuke NISHIHARA, Tokyo (JP); Masahiro KUBO, Tokyo (JP)

(73) Assignee: NEC Corporation, Minato-ku, Tokyo (JP)

(21) Appl. No.: 18/848,655

(22) PCT Filed: Mar. 29, 2022
(86) PCT No.: PCT/JP2022/015449

§ 371 Date: Sep. 19, 2024

(51) Int. Cl. G08B 25/01; G08B 21/02; G16H 10/20; G16H 10/60; G16H 40/67; G16H 50/30

(52) U.S. Cl. CPC G08B 25/016; G08B 21/02; G16H 10/20; G16H 10/60; G16H 40/67; G16H 50/30

(57) ABSTRACT

A notification apparatus of the present invention includes: a detecting unit that detects a degree of anomaly of a person from electrocardiogram data of the person measured with a wearable device; a determining unit that determines a level of emergency of an anomaly of the person based on the degree of anomaly; a selecting unit that selects, from among response data representing a content of a response to be notified to the person or to a third party set in advance, the response data in accordance with the level of emergency; and a notifying unit that provides a notification corresponding to the selected response data. The notification apparatus of the present invention can support user's decision-making, for example.

### TECHNICAL FIELD

The present invention relates to a notification method, a notification apparatus, and a program.

### BACKGROUND ART

One method for diagnosing a physical condition is to use an electrocardiogram. An electrocardiogram is generally measured using an electrocardiogram a medical institution, but in recent years, it is also measured using a wearable device such as a wristwatch-type portable terminal. Then, since a wearable device is worn by a person at all times, it is possible to measure an electrocardiogram at all times. For this reason, Patent Literature 1 describes determining the degree of anomaly of a body from an electrocardiogram measured with a wearable device and transmitting notification information to a notification destination registered in advance corresponding to the degree of anomaly.

### CITATION LIST

### Patent Literature

Patent literature 1: Japanese Unexamined Patent Application Publication No. JP-A 2020-130772

### SUMMARY OF INVENTION

### Technical Problem

However, in the method described in Patent Literature 1, a notification destination of notification information is previously set in accordance with the degree of anomaly of a body determined from an electrocardiogram, and the condition of a person and other circumstances are not taken into consideration. In addition, the type of an electrocardiogram that can be measured with a wearable device is limited, and the degree of anomaly cannot necessarily be determined in accordance with the condition of a person. This causes a problem that it is not clear whether a notification destination of notification information and a notification itself are appropriate and, even when an anomaly is detected from an electrocardiogram measured with a wearable device, a notification appropriate to the condition of a person may not be provided.

An object of the present invention is to provide a notification method which can solve the abovementioned problem that when an anomaly is detected from an electrocardiogram measured with a wearable device, a notification appropriate to the condition of a person may not be provided.

### Solution to Problem

A notification method as an aspect of the present invention includes: detecting a degree of anomaly of a person from electrocardiogram data of the person measured with a wearable device; determining a level of emergency of an anomaly of the person based on the degree of anomaly; selecting, from among response data representing a content of a response to be notified to the person or to a third party set in advance, the response data in accordance with the level of emergency; and providing a notification corresponding to the selected response data.

Further, a notification apparatus as an aspect of the present invention includes: a detecting unit that detects a degree of anomaly of a person from electrocardiogram data of the person measured with a wearable device; a determining unit that determines a level of emergency of an anomaly of the person based on the degree of anomaly; a selecting unit that selects, from among response data representing a content of a response to be notified to the person or to a third party set in advance, the response data in accordance with the level of emergency; and a notifying unit that provides a notification corresponding to the selected response data.

Further, a computer program as an aspect of the present invention includes instructions for causing a computer to execute processes to: detect a degree of anomaly of a person from electrocardiogram data of the person measured with a wearable device; determine a level of emergency of an anomaly of the person based on the degree of anomaly; select, from among response data representing a content of a response to be notified to the person or to a third party set in advance, the response data in accordance with the level of emergency; and provide a notification corresponding to the selected response data.

### Advantageous Effects of Invention

Configured as described above, the present invention enables notification appropriate to the condition of a person when an anomaly is detected from an electrocardiogram measured with a wearable device.

### BRIEF DESCRIPTION OF DRAWINGS

FIG. 1 is a view showing the overall configuration of a notification system in a first example embodiment of the present invention.

FIG. 2 is a block diagram showing the configuration of a user terminal disclosed in FIG. 1.

FIG. 3 is a flowchart showing the operation of the user terminal disclosed in FIG. 1.

FIG. 4 is a view showing the overall configuration of a notification system in a second example embodiment of the present invention.

FIG. 5 is a block diagram showing the configuration of the user terminal disclosed in FIG. 4.

FIG. 6 is a block diagram showing the hardware configuration of a notification apparatus in a third example embodiment of the present invention.

FIG. 7 is a block diagram showing the configuration of the notification apparatus in the third example embodiment of the present invention.

FIG. 8 is a flowchart showing the operation of the notification apparatus in the third example embodiment of the present invention.

### DESCRIPTION OF EXAMPLE EMBODIMENTS

### First Example Embodiment

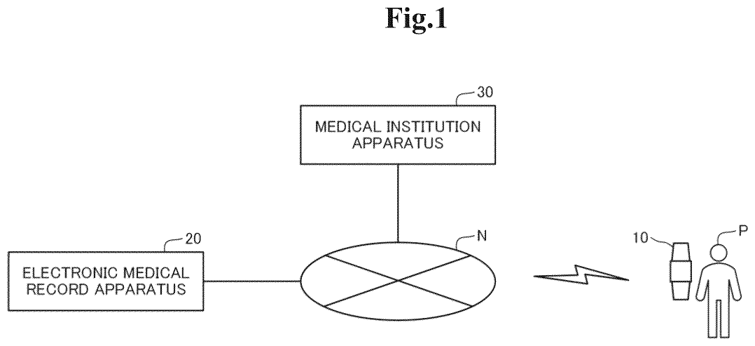

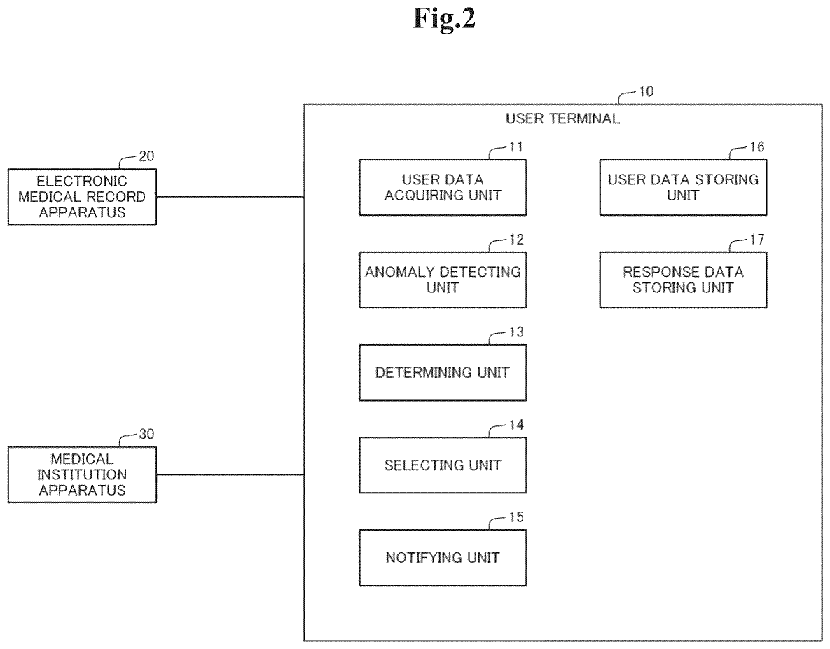

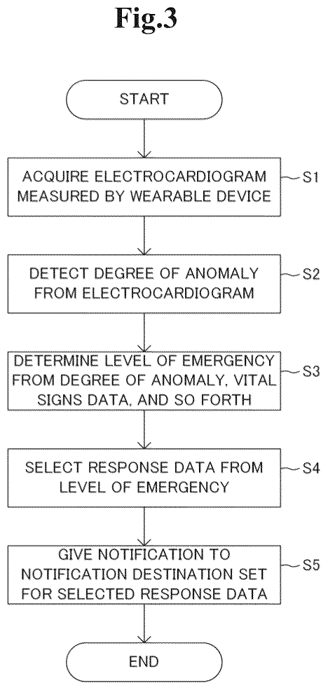

A first example embodiment of the present invention will be described with reference to FIGS. 1 to 3. FIGS. 1 and 2 are views for describing the configuration of a notification system, and FIG. 3 is a view for describing the processing operation of the notification system.

### [Configuration]

The notification system of the present invention is for providing a notification appropriate to the condition of a person P when a physical anomaly of the person P is detected from an electrocardiogram measured with a wearable device. For this reason, as shown in FIG. 1, a user terminal 10, which is a wearable device in this example embodiment, is a wristwatch-type portable terminal worn on the arm of the person P and is equipped with an electrocardiogrameasures an electrocardiogram. However, the user terminal 10 that is a wearable device is not limited to being a wristwatch-type portable terminal, and may be any device that can be worn by the person P to measure an electrocardiogram. In addition, the user terminal 10 may be a combination of a wearable device that is a wristwatch-type portable terminal and a portable information processing terminal such as a smartphone carried by the person P and connected to the wearable device via short-range wireless communication.

As shown in FIG. 1, the notification system includes the user terminal 10, which is a wearable device that can be connected to a network N. Then, the user terminal 10 is connected to an electronic medical record apparatus 20 and a medical institution apparatus 30 via the network N.

The electronic medical record apparatus 20 is an information processing apparatus managed by a medical institution, and stores an electronic medical record of the person P. For example, the examination result and diagnosis result of the person P are recorded in the electronic medical record. As an example, in the electronic medical record, the following data of the person P are recorded: basic physical data such as age, gender, height, and weight; measurement data such as heart rate, body temperature, blood pressure, and electrocardiogram; and medical condition data such as state of consciousness, current or past disease, condition at the time of diagnosis, and condition at the time of examination. Then, as will be described later, the electronic medical record apparatus 20 has a function of transmitting data recorded in the electronic medical record of the person P wearing the user terminal 10 to the user terminal 10 in response to a request from the user terminal 10.

The medical institution apparatus 30 is an information processing apparatus managed by a medical institution, and can be operated by the staff of the medical institution such as a doctor and an examiner. As will be described later, the medical institution apparatus 30 receives a notification from the user terminal 10 and outputs the content of the notification so as to inform the doctor, the examiner, and the like.

The user terminal 10 is a wearable device including an arithmetic logic unit and a memory unit and, as described above, includes an electrocardiogrameasures an electrocardiogram from the person P. The user terminal 10 also includes various sensors that measure vital signs data indicating the physical condition of the person P, such as body temperature, blood pressure, and oxygen saturation.

Then, as shown in FIG. 2, the user terminal 10 includes a user data acquiring unit 11, an anomaly detecting unit 12, a determining unit 13, a selecting unit 14, and a notifying unit 15. The respective functions of the user data acquiring unit 11, the anomaly detecting unit 12, the determining unit 13, the selecting unit 14, and the notifying unit 15 can be realized by the arithmetic logic unit executing a program for realizing the respective functions stored in the memory unit. The user terminal 10 also includes a user data storing unit 16 and a response data storing unit 17. The user data storing unit 16 and the response data storing unit 17 are configured with the memory unit. The respective components will be described in detail below.

The user data acquiring unit 11 acquires electrocardiogram data measured by an electrocardiogramnstalled in the user terminal 10 and stores the electrocardiogram data into the user data storing unit 16. For example, the user data acquiring unit 11 may acquire electrocardiogram data automatically measured at a preset timing such as a fixed time interval, or may acquire electrocardiogram data measured in response to an instruction from the person P. Moreover, the user data acquiring unit 11 acquires vital signs data indicating physical condition such as body temperature, blood pressure and oxygen saturation measured by various sensors installed in the user terminal 10, and stores the data into the user data storing unit 16. The user data acquiring unit 11 acquires, as vital signs data, numerical values input by the person P and text information indicating the condition or symptom at that time, and stores them into the user data storing unit 16. At this time, the user data acquiring unit 11 acquires data input by the person P through keyboard input, voice input, or the like.

Furthermore, the user data acquiring unit 11 sends a request the electronic medical record apparatus 20 to acquire any record data recorded in the electronic medical record of the person P, and stores the record data into the user data storing unit 16. For example, the user data acquiring unit 11 also acquires the following data recorded in the electronic medical record of the person P: basic physical data such as age, gender, height, and weight; measurement data such as heart rate, body temperature, blood pressure, and electrocardiogram; examination data such as blood test result and image diagnosis result; and medical condition data such as state of consciousness, current or past disease, condition at the time of diagnosis, and condition at the time of examination. The user data acquiring unit 11 may acquire the abovementioned record data by accepting an input from the person P. The acquired record data may also include genetic data, lifestyle, external environment, family information and so forth of the person P.

As described above, the record data acquired by the user data acquiring unit 11 may be acquired, for example, via the portable information processing terminal connected to the wearable device via short-range wireless communication.

The anomaly detecting unit 12 (detecting unit) detects the degree of anomaly of the person P from the electrocardiogram data acquired as described above. Specifically, the anomaly detecting unit 12 detects the degree of anomaly representing the level of being a specific disease such as myocardial infarction based on changes in detection value and waveform that can be detected from electrocardiogram data. As an example, the anomaly detecting unit 12 detects the degree of anomaly from changes in various detection values and waveforms such as P wave, PQ interval, QRS wave, and ST height from electrocardiogram data. The anomaly detecting unit 12 may use an evaluation model generated in advance through machine learning and detect the degree of anomaly that is output by inputting electrocardiogram data into the evaluation model. For example, the evaluation model is a model obtained by learning normal electrocardiograms and anomalous electrocardiograms of various diseases such as myocardial infarction, and has been trained to output a higher degree of anomaly as the input electrocardiogram data shows a more diseased state.

Further, the anomaly detecting unit 12 calculates the accuracy of the degree of anomaly in the electrocardiogram data detected as described above, that is, the likelihood (probability) of the degree of anomaly. For example, the anomaly detecting unit 12 calculates the likelihood using a likelihood function generated in advance based on statistics of the degree of anomaly. However, the anomaly detecting unit 12 may calculate the value of the probability of the degree of anomaly by any method.

The determining unit 13 determines the level of emergency of the anomaly of the person P based on the degree of anomaly detected from the electrocardiogram data as described above. Specifically, the determining unit 13 determines that there is an emergency when the degree of anomaly is equal to or more than a preset threshold value, and determines that there is no emergency when the degree of anomaly is less than the preset threshold value. At this time, the determining unit 13 may further considers vital signs data such as the body temperature, blood pressure and oxygen saturation of the person P acquired as descried above, record data as recorded in the electronic medical record, the likelihood and so forth to determine the level of emergency of the anomaly of the person P. As an example, in a case where, although it may be determined that there is an emergency from the degree of anomaly as described above, the degree of anomaly does not significantly exceed the threshold value and it cannot be determined to be anomalous from the vital signs data or the record data, the determining unit 13 ultimately determines that there is no emergency. Moreover, as another example, in a case where, although it may be determined that there is an emergency from the degree of anomaly as described above, it can be determined that an anomaly is hard to arise in consideration of age and body shape or the likelihood is lower than a preset criterion, the determining unit 13 ultimately determines that there is no emergency. Such determination is made using a trained model or a determination formula generated in advance through learning using the degree of anomaly, vital signs data, record data, and likelihood.

However, the determining unit 13 is not limited to determining the level of emergency of the anomaly of the person P as emergency or no emergency as described above, and may determine the level of emergency in a plurality of stages. For example, the determining unit 13 may use a model or a determination formula to determine the level of emergency in five stages from the degree of anomaly, vital signs data, record data, and the value of the likelihood.

The selecting unit 14 selects response data from among prepared response data based on the level of emergency determined as described above. Here, the response data prepared and stored in the response data storing unit 17 includes data to be notified to the person P indicating the content of response by the person P, and data to be notified to a third party such as a medical institution indicating the content of response by the third party, as will be descried below. For example, “Contact the emergency service”, “Contact the family doctor”, and “Observation” are prepared as the response data to be notified to the person P, and “Contact the specialist”, “Acquire a 12-lead electrocardiogram”, and “Observation” are prepared as the response data to be notified to a third party.”

Then, the selecting unit 14 selects one or a plurality of response data from among the response data as descried above based on the determined level of emergency. At this time, the selecting unit 14 may select response data also based on the value of the degree of anomaly, vital signs data, record data, likelihood, and the like. For example, when there is no emergency, the selecting unit 14 selects “Observation” of the response data to be notified to the person P. On the other hand, when there is an emergency, the selecting unit 14 selects “Contact the family doctor” of the response data to be notified to the person P or “Acquire a 12-lead electrocardiogram” of the response data to be notified to a third party. As another example, in a case where there is an emergency and also the degree of anomaly is high or the record data includes a history of heart disease, the selecting unit 14 selects “Contact the emergency service” of the response data to be notified to the person P and “Contact the specialist” of the response data to be notified to a third party.

In a case where the level of emergency of the anomaly of the person P is determined in a plurality of stages by the abovementioned determining unit 13, the selecting unit 14 may select one or more of the abovementioned response data in accordance with the stage. The abovementioned response data is merely an example, and response data with other contents may be prepared so as to be selectable.

The notifying unit 15 provides a notification based on the content of the response data selected by the selecting unit 14 as described above. For example, in a case where response data to be notified to the person P is selected, the notifying unit 15 displays, on a display screen of the wearable device serving as the user terminal 10, any of “Contact the emergency service”, “Contact the family doctor”, and “Observation”. At this time, the notifying unit 15 may output the information by voice from the wearable device, or may display the information on a display screen of the portable information processing terminal connected to the wearable device via short-range wireless communication. In addition, in a case where response data to be notified to a medical institution that is a third party is selected, the notifying unit 15 transmits, to the address of the medical institution apparatus 30 registered in advance, any information of “Contact the specialist”, “Acquire a 12-lead electrocardiogram”, and “Observation”.

### [Operation]

Next, the operation of the above user terminal 10 will be described mainly with reference to a flowchart of FIG. 3.

The user terminal 10 acquires electrocardiogram data measured by an electrocardiograph installed in the user terminal 10 (step S1). Then, the user terminal 10 detects the degree of anomaly of a person P from the acquired electrocardiogram data (step S2). For example, the user terminal 10 may detect the degree of anomaly representing the level of being a specific disease such as myocardial infarction based on changes in a detection value and a waveform that can be detected from the electrocardiogram data, or may detect using an evaluation model. The acquisition of electrocardiogram data and the detection of the degree of anomaly described above are performed at constant time intervals at all times.

Next, the user terminal 10 determines the level of emergency based on the detected degree of anomaly (step S3). For example, the user terminal 10 determines that there is an emergency when the degree of anomaly is equal to or more than a preset threshold value, and determines that there is no emergency when the degree of anomaly is less than the preset threshold value. At this time, the user terminal 10 may determine the level of emergency using not only the degree of anomaly but also vital signs data acquired from the electronic medical record apparatus 20 or acquired by the user terminal 10 itself.

Next, the user terminal 10 selects response data from among prepared response data based on the determined level of emergency (step S4). For example, the prepared response data includes response data to be notified to the person P representing the content of a response by the person P and response data to be notified to a third party such as a medical institution representing the content of a response by the third party. Therefore, as an example, in the case of determining that there is an emergency, the user terminal 10 may select “Contact the family doctor” of the response data to be notified to the person P or “Acquire a 12-lead electrocardiogram” of the response data to be notified to a third party. At this time, the user terminal 10 may select response data based on not only the level of emergency but also the value of the degree of anomaly, vital signs data, record data, likelihood, and so forth. As an example, in a case where, when there is an emergency, the degree of anomaly is high or record data includes the history of a cardiac disease, the user terminal 10 may select “Contact the emergency service” of the response data to be notified to the person P and “Contact the specialist” of the response data to be notified to a third party.

After that, the user terminal 10 provides a notification based on the content of the selected response data (step S5). For example, the user terminal 10 displays a response such as “Contact the emergency service” on a display screen of a wearable device that is the user terminal 10, or display a response such as “Acquire a 12-lead electrocardiogram” to the address of the medical institution apparatus 30 registered in advance.

As described above, according to this example embodiment, the level of emergency is determined from the degree of anomaly detected from electrocardiogram data of the person P, a response by the person P or a response by a third party is selected based on the level of emergency, and a notification is provided to a person who will respond.

### Second Example Embodiment

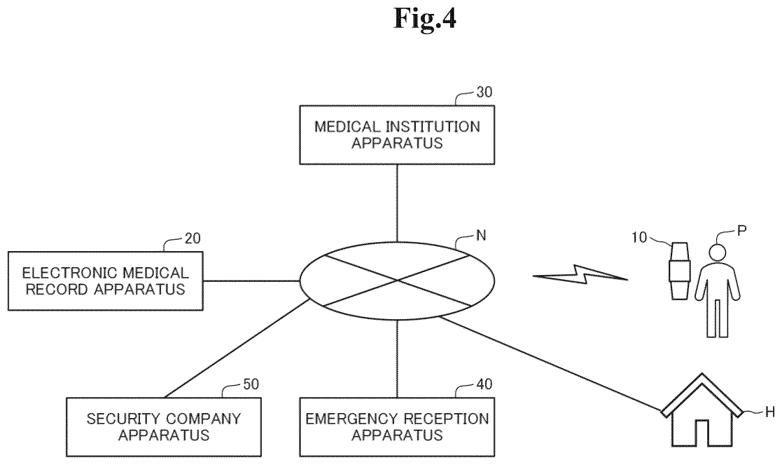

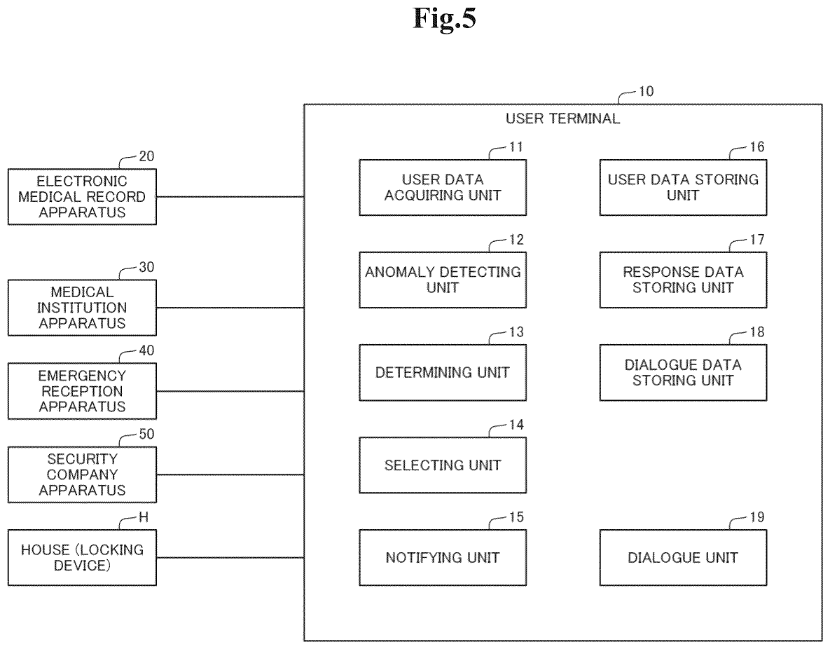

Next, a second example embodiment of the present invention will be described with reference to FIGS. 4 and 5. FIGS. 4 and 5 are views for describing the configuration of a notification system in the second example embodiment.

The notification system in this example embodiment has a configuration similar to the configuration shown in FIGS. 1 and 2 described in the first example embodiment above. Meanwhile, the notification system in this example embodiment also has the following configuration in addition to the configuration of the first example embodiment. Below, the configuration different from the configuration of the first example embodiment will be described mainly.

First, as shown in FIG. 4, in the notification system in this example embodiment, a locking device installed on a door such as the entrance of a house H of a person P is connected to a network N and is configured so that it can be unlocked remotely. Moreover, an emergency reception apparatus 40, which is an information processing apparatus of an emergency service, and a security company apparatus 50, which is an information processing apparatus of a security company, are connected to the network N and are configured to be able to receive notifications from the user terminal 10.

Then, as shown in FIG. 5, the user terminal 10 further includes a dialogue unit 19 realized by an arithmetic logic unit executing a program. The user terminal 10 also includes a dialogue data storing unit 18 formed in a memory unit. The respective components will be described in detail below.

When the level of emergency determined by the abovementioned determining unit 13 is lower than a preset criterion, for example, when it is determined that there is no emergency, the dialogue unit 19 outputs medical interview data representing the content of a medical interview to the person P. As an example, in a case where, although the degree of anomaly is high and above a threshold value, it is determined that there is no emergency in consideration of vital signs data, record data, likelihood and so forth, the dialogue unit 19 outputs medical interview data selected from medical interview data stored in the dialogue data storing unit 18 by displaying on a display unit of the user terminal 10 or by emitting by voice. The medical interview data stored in the dialogue data storing unit 18 may be, for example, questions for inquiring about the condition of the person P, such as “How is your condition?” and “Are you having difficulty breathing?”, but may be any medical interview content. The dialogue unit 19 may change the content of medical interview to be selected in accordance with the contents of the vital signs data and electrocardiogram data acquired as described above, for example. As an example, in a case where an initial value to be selected is set to “How is your condition?”, the dialogue unit 19 may change the content of medical interview to “Are you having difficulty breathing?” when the vital signs data indicates a high heart rate or an arrhythmia is detected.

Then, after outputting the medical interview data to the person P as described above, the dialogue unit 19 acquires answer data representing an answer to the medical interview data input from the person P into the user terminal 10. For example, the dialogue unit 19 acquires answer data input by the person P through voice input, keyboard input, or the like. As an example, in the case of acquiring answer data such as “I feel short of breath” from the person P, the dialogue unit 19 notifies the answer data to the determining unit 13. In response to this, the determining unit 13 performs determination of the level of emergency again in consideration of the answer data. For example, in the case of acquiring answer data determined to be a high level of emergency set in advance, the determining unit 13 determines that the level of emergency is higher than the previous time.

Further, in a case where the level of emergency determined by the determining unit 13 is higher than the preset criterion, for example, in a case where it is determined that there is an emergency, the dialogue unit 19 outputs information requesting consent to providing physical data of the person P to a third party by displaying it on the display unit of the user terminal 10 or emitting a voice. Then, after outputting an inquiry for consent to the person P as described above, the dialogue unit 19 acquires the presence or absence of consent input from the person P into the user terminal 10, and notifies the presence or absence of consent to the notifying unit 15. For example, the dialogue unit 19 acquires the presence or absence of consent input by the person P through voice input, keyboard input or the like, and notifies it to the notifying unit 15.

In the case of obtaining consent to provide physical data to a third party from the person P as described above, the notifying unit 15 notifies physical data such as electrocardiogram data and vital signs data of the person P acquired by the user terminal 10 to the medical institution apparatus 30. At this time, as described above, in the case of providing a notification to the address of the medical institution apparatus 30 registered in advance in accordance with the selected response data, the notifying unit 15 notifies physical data of the person P along with the content of the response data.

Further, the response data storing unit 17 in this example embodiment may contain, as response data to be notified to a third party, response data to be notified to the emergency reception apparatus 40 of the emergency service and response data to be notified to the security company apparatus 50 of the security company. Consequently, the selecting unit 14 may select response data to be notified to the emergency service and the security company as described above based on the determined level of emergency, the value of the degree of anomaly, vital signs data, record data, likelihood and so forth in the same manner as described above. Then, the notifying unit 15 can also provide a notification to the emergency reception apparatus 40 and the security company apparatus 50 in accordance with the response data.

Furthermore, in this example embodiment, in a case where the level of emergency determined by the determining unit 13 is higher than the preset criterion, for example, in a case where it is determined that there is an emergency, the notifying unit 15 may transmit a command to remotely unlock the locking device of the house H of the person P via the network N. At this time, the notifying unit 15 may acquire location information of a wearable device serving as the user terminal 10 and, in a case where the person P is located in the house H, transmit a command to remotely unlock. Consequently, even when the person P is unable to move in the house H, the locking device can be unlocked and a rescue team or the like can rescue.

The processing functions and memory units included by the user terminal 10 described in the first and second example embodiments described above are not necessarily limited to being installed in the user terminal 10, and some of them may be installed in an information processing apparatus such as a server connected to the user terminal 10 via a network. That is to say, the information processing by the user terminal 10 described above is not limited to being executed entirely on the user terminal 10, and some of the processing may be executed on the server. For example, the user terminal 10 may transmit vital signs data such as an electrocardiogram acquired by the user data acquiring unit 11 to the server, and the server may perform anomaly detection by the anomaly detecting unit 12, determination of the level of emergency by the determining unit 13, and further selection of response data by the selecting unit 14. Then, the selected response data may be transmitted from the server to the user terminal 10, and the response data may be displayed or notified by the notifying unit 15 on the user terminal 10.

### Third Example Embodiment

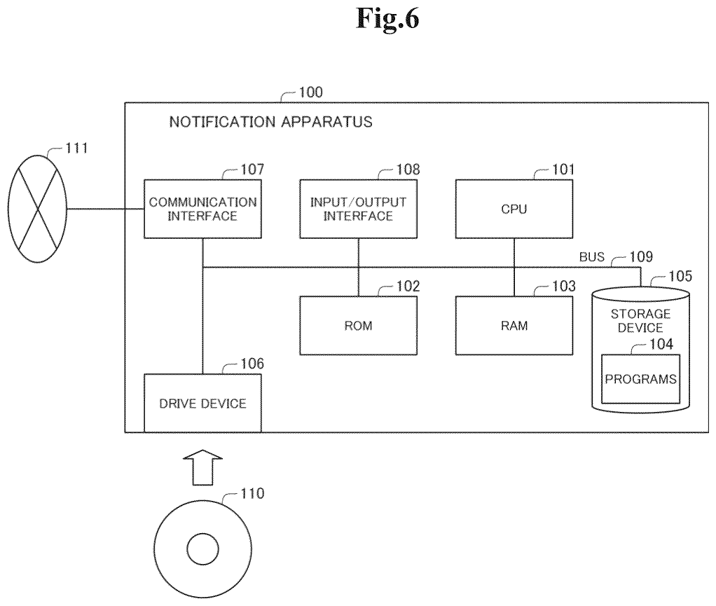

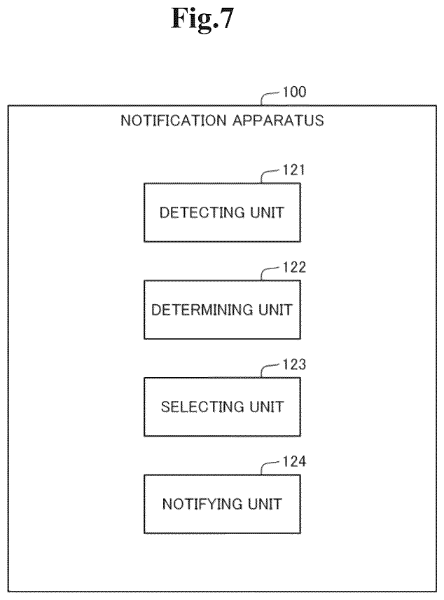

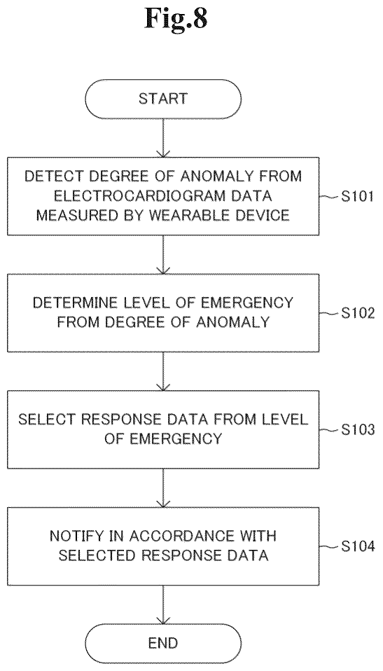

Next, a third example embodiment of the present invention will be described with reference to FIGS. 6 to 8. FIGS. 6 and 7 are block diagrams showing the configuration of a notification apparatus in the third example embodiment, and FIG. 8 is a flowchart showing the operation of the notification apparatus. In this example embodiment, the overview of the configurations of the notification apparatus and the notification method described in the example embodiments above is shown.

First, with reference to FIG. 6, the hardware configuration of a notification apparatus 100 in this example embodiment will be described. The notification apparatus 100 is configured with a general information processing apparatus and, as an example, has the following hardware configuration including:
a CPU (Central Processing Unit) 101 (arithmetic logic unit) (or a GPU (Graphics Processing Unit));
a ROM (Read Only Memory) 102 (memory unit);
a RAM (Random Access Memory) 103 (memory unit);
programs 104 loaded to the RAM 103;
a storage device 105 storing the programs 104;
a drive device 106 that reads from and writes into a storage medium 110 outside the information processing apparatus;
a communication interface 107 connected to a communication network 111 outside the information processing apparatus;
an input/output interface 108 that inputs and outputs data; and
a bus 109 that connects the respective components.

Then, the notification apparatus 100 can construct and include a detecting unit 121, a determining unit 122, a selecting unit 123, and a notifying unit 124 shown in FIG. 7 by acquisition and execution of the programs 104 by the CPU 101. The programs 104 are, for example, stored in advance in the storage device 105 or the ROM 102, and are loaded into the RAM 103 and executed by the CPU 101 as necessary. In addition, the programs 104 may be provided to the CPU 101 via the communication network 111, or the programs 104 may be stored in the storage medium 110 in advance and read out by the drive device 106 and provided to the CPU 101. However, the detecting unit 121, the determining unit 122, the selecting unit 123, and the notifying unit 124 described above may be constructed using dedicated electronic circuits for realizing such means.

FIG. 6 shows an example of the hardware configuration of the information processing apparatus serving as the notification apparatus 100, and the hardware configuration of the information processing apparatus is not limited to the above case. For example, the information processing apparatus may be configured with part of the above configuration, such as without the drive device 106.

Then, the notification apparatus 100 executes a notification method shown in the flowchart of FIG. 8 by the functions of the detecting unit 121, the determining unit 122, the selecting unit 123, and the notifying unit 124 constructed by the program as described above.

As shown in FIG. 8, the notification apparatus 100 executes processes to:
detect a degree of anomaly of a person from electrocardiogram data of the person measured by a wearable device (step S101);
determine a level of emergency of the anomaly of the person based on the degree of anomaly (step S102);
select, from among preset response data representing a content of response to be notified to the person or a third party, response data in accordance with the level of emergency (step S103); and
provide a notification according to the selected response data (step S104).

Configured as described above, the present invention enables determination of the level of emergency from the degree of anomaly detected from the electrocardiogram data of a person, selection of response by the person or response by a third party based on the level of emergency, and notification to a person who will respond. Consequently, a notification appropriate to the condition of a person is sent.

The abovementioned programs can be stored and provided to a computer using various types of non-transitory computer-readable mediums. Non-transitory computer-readable mediums include various types of tangible storage mediums. Examples of non-transitory computer-readable mediums include a magnetic recording medium (e.g., floppy disk, magnetic tape, hard disk drive), a magneto-optical recording medium (e.g., magneto-optical disk), a CD-ROM (Read Only Memory), a CD-R, a CD-R/W, and a semiconductor memory (e.g., mask ROM, PROM (Programmable ROM), EPROM (Erasable PROM), flash ROM, and RAM (Random Access Memory)). The program may also be provided to a computer by various types of transitory computer-readable mediums. Examples of transitory computer-readable mediums include an electrical signal, an optical signal, and an electromagnetic wave. The temporary computer-readable medium can provide the program to the computer via a wired communication path such as an electric wire or an optical fiber, or via a wireless communication path.

Although the present invention has been described above with reference to the above example embodiments, the present invention is not limited to the above example embodiments. The configuration and details of the present invention can be modified in various ways that are understandable to a person skilled in the art within the scope of the present invention. Moreover, at least one or more of the functions of the detecting unit 121, the determining unit 122, the selecting unit 123 and the notifying unit 124 described above may be executed by an information processing apparatus installed and connected anywhere on the network, that is, may be executed by so-called cloud computing.

### Supplementary Notes

The whole or part of the example embodiments disclosed above can be described as the following supplementary notes. The overview of the configurations of a notification method, a notification apparatus, and a program according to the present invention will be described below. However, the present invention is not limited to the following configurations.

### (Supplementary Note 1)

A notification method comprising:
detecting a degree of anomaly of a person from electrocardiogram data of the person measured with a wearable device;
determining a level of emergency of an anomaly of the person based on the degree of anomaly;
selecting, from among response data representing a content of a response to be notified to the person or to a third party set in advance, the response data in accordance with the level of emergency; and
providing a notification corresponding to the selected response data.

### (Supplementary Note 2)

The notification method according to Supplementary Note 1, comprising
selecting the response data in accordance with the level of emergency from among the response data including the response data representing a content of a response by the person and the response data representing a content of a response by the third party.

### (Supplementary Note 3)

The notification method according to Supplementary Note 2, comprising
selecting the response data in accordance with the level of emergency from among the response data including the response data representing a content of a response in which the person contacts a medical institution and the response data representing a content of a response in which the medical institution serving as the third party measures an electrocardiogram of the person.

### (Supplementary Note 4)

The notification method according to any one of Supplementary Notes 1 to 3, comprising
determining the level of emergency of the anomaly of the person based on the degree of anomaly and on record data related to a body of the person.

s(Supplementary Note 5)

The notification method according to any one of Supplementary Notes 1 to 4, comprising
determining the level of emergency of the anomaly of the person based on a physical condition of the person acquired with the wearable device.

### (Supplementary Note 6)

The notification method according to any one of Supplementary Notes 1 to 5, comprising:
calculating likelihood of the degree of anomaly detected from the electrocardiogram data; and
determining the level of emergency of the anomaly of the person based on the likelihood.

### (Supplementary Note 7)

The notification method according to any one of Supplementary Notes 1 to 6, comprising:
in a case where the level of emergency is lower than a preset criterion, outputting medical interview data representing a content of a medical interview to the person, and acquiring answer data representing an answer to the medical interview data input from the person; and
determining the level of emergency based on the answer data.

### (Supplementary Note 8)

The notification method according to Supplementary Note 7, comprising
outputting the medical interview data with the content of the medical interview changed based on a physical condition of the person acquired with the wearable device.

### (Supplementary Note 9)

The notification method according to any one of Supplementary Notes 1 to 8, comprising:
in a case where the level of emergency is higher than a preset criterion, receiving a consent by the person to provide data related to a body of the person to the third party; and
notifying the data related to the body of the person to the third party when receiving the consent.

### (Supplementary Note 10)

The notification method according to any one of Supplementary Notes 1 to 9, comprising
performing a process to unlock a place in which the person is located in a case where the level of emergency is higher than a preset criterion.

### (Supplementary Note 11)

A notification apparatus comprising:
a detecting unit that detects a degree of anomaly of a person from electrocardiogram data of the person measured with a wearable device;
a determining unit that determines a level of emergency of an anomaly of the person based on the degree of anomaly;
a selecting unit that selects, from among response data representing a content of a response to be notified to the person or to a third party set in advance, the response data in accordance with the level of emergency; and
a notifying unit that provides a notification corresponding to the selected response data.

### (Supplementary Note 12)

The notification apparatus according to Supplementary Note 11, wherein
the selecting unit selects the response data in accordance with the level of emergency from among the response data including the response data representing a content of a response by the person and the response data representing a content of a response by the third party.

### (Supplementary Note 13)

The notification apparatus according to Supplementary Note 12, wherein
the selecting unit selects the response data in accordance with the level of emergency from among the response data including the response data representing a content of a response in which the person contacts a medical institution and the response data representing a content of a response in which the medical institution serving as the third party measures an electrocardiogram of the person.

### (Supplementary Note 14)

The notification apparatus according to any one of Supplementary Notes 11 to 13, wherein
the determining unit determines the level of emergency of the anomaly of the person based on the degree of anomaly and on record data related to a body of the person.

### (Supplementary Note 15)

The notification apparatus according to any one of Supplementary Notes 11 to 14, wherein
the determining unit determines the level of emergency of the anomaly of the person based on a physical condition of the person acquired with the wearable device.

### (Supplementary Note 16)

The notification apparatus according to any one of Supplementary Notes 11 to 15, wherein:
the detecting unit calculates likelihood of the degree of anomaly detected from the electrocardiogram data; and
the determining unit determines the level of emergency of the anomaly of the person based on the likelihood.

### (Supplementary Note 17)

The notification apparatus according to any one of Supplementary Notes 11 to 16, comprising
a dialogue unit that, in a case where the level of emergency is lower than a preset criterion, outputs medical interview data representing a content of a medical interview to the person, and acquires answer data representing an answer to the medical interview data input from the person, wherein
the determining unit determines the level of emergency based on the answer data.

### (Supplementary Note 18)

The notification apparatus according to Supplementary Note 17, wherein
the dialogue unit outputs the medical interview data with the content of the medical interview changed based on a physical condition of the person acquired with the wearable device.

### (Supplementary Note 19)

The notification apparatus according to any one of Supplementary Notes 11 to 18, comprising:
a dialogue unit that, in a case where the level of emergency is higher than a preset criterion, receives a consent by the person to provide data related to a body of the person to the third party, wherein
the notifying unit notifies the data related to the body of the person to the third party when receiving the consent.

### (Supplementary Note 20)

The notification apparatus according to any one of Supplementary Notes 11 to 19, wherein
the notifying unit performs a process to unlock a place in which the person is located in a case where the level of emergency is higher than a preset criterion.

### (Supplementary Note 21)

A non-transitory computer-readable storage medium in which a program is stored, the program comprising instructions for causing a computer to execute processes to:
detect a degree of anomaly of a person from electrocardiogram data of the person measured with a wearable device;
determine a level of emergency of an anomaly of the person based on the degree of anomaly;
select, from among response data representing a content of a response to be notified to the person or to a third party set in advance, the response data in accordance with the level of emergency; and
provide a notification corresponding to the selected response data.

### REFERENCE SIGNS LIST

10 user terminal
11 user data acquiring unit
12 anomaly detecting unit
13 determining unit
14 selecting unit
15 notifying unit
16 user data storing unit
17 response data storing unit
18 dialogue data storing unit
19 dialogue unit
20 electronic medical record apparatus
30 medical institution apparatus
40 emergency reception apparatus
50 security company apparatus
P person
H house
100 notification apparatus
101 CPU
102 ROM
103 RAM
104 programs
105 storage device
106 drive device
107 communication interface
108 input/output interface
109 bus
110 storage medium
111 communication network
121 detecting unit
122 determining unit
123 selecting unit
124 notifying unit

What is claimed is:

1. A notification method comprising:
detecting a degree of anomaly of a person from electrocardiogram data of the person measured with a wearable device;
determining a level of emergency of an anomaly of the person based on the degree of anomaly;
selecting, from among response data representing a content of a response to be notified to the person or to a third party set in advance, the response data in accordance with the level of emergency; and
providing a notification corresponding to the selected response data.

2. The notification method according to claim 1, comprising
selecting the response data in accordance with the level of emergency from among the response data including the response data representing a content of a response by the person and the response data representing a content of a response by the third party.

3. The notification method according to claim 2, comprising selecting the response data in accordance with the level of emergency from among the response data including the response data representing a content of a response in which the person contacts a medical institution and the response data representing a content of a response in which the medical institution serving as the third party measures an electrocardiogram of the person.

4. The notification method according to claim 1, comprising
determining the level of emergency of the anomaly of the person based on the degree of anomaly and on record data related to a body of the person.

5. The notification method according to claim 1, comprising
determining the level of emergency of the anomaly of the person based on a physical condition of the person acquired with the wearable device.

6. The notification method according to claim 1, comprising:
calculating likelihood of the degree of anomaly detected from the electrocardiogram data; and
determining the level of emergency of the anomaly of the person based on the likelihood.

7. The notification method according to claim 1, comprising:
in a case where the level of emergency is lower than a preset criterion, outputting medical interview data representing a content of a medical interview to the person, and acquiring answer data representing an answer to the medical interview data input from the person; and
determining the level of emergency based on the answer data.

8. The notification method according to claim 7, comprising outputting the medical interview data with the content of the medical interview changed based on a physical condition of the person acquired with the wearable device.

9. The notification method according to claim 1, comprising:
in a case where the level of emergency is higher than a preset criterion, receiving a consent by the person to provide data related to a body of the person to the third party; and
notifying the data related to the body of the person to the third party when receiving the consent.

10. The notification method according to claim 1, comprising
performing a process to unlock a place in which the person is located in a case where the level of emergency is higher than a preset criterion.

11. A notification apparatus comprising:
at least one memory storing processing instructions; and
at least one processor configured to execute the processing instructions to:
detect a degree of anomaly of a person from electrocardiogram data of the person measured with a wearable device;
determine a level of emergency of an anomaly of the person based on the degree of anomaly;
select, from among response data representing a content of a response to be notified to the person or to a third party set in advance, the response data in accordance with the level of emergency; and
provide a notification corresponding to the selected response data.

12-20. (canceled)

21. A non-transitory computer-readable storage medium in which a program is stored, the program comprising instructions for causing a computer to execute processes to:
detect a degree of anomaly of a person from electrocardiogram data of the person measured with a wearable device;
determine a level of emergency of an anomaly of the person based on the degree of anomaly;
select, from among response data representing a content of a response to be notified to the person or to a third party set in advance, the response data in accordance with the level of emergency; and
provide a notification corresponding to the selected response data.

22. The notification method according to claim 1, comprising
detecting the degree of anomaly of the person using an evaluation model generated through machine learning.
